(19) United States
(12) Patent Application Publication
(10) Pub. No.: US 2024/0170135 A1
(43) Pub. Date: May 23, 2024

(54) LOAD BALANCING IN EXAM ASSIGNMENTS FOR EXPERT USERS WITHIN A RADIOLOGY OPERATIONS COMMAND CENTER (ROCC) STRUCTURE

(71) Applicant: KONINKLIJKE PHILIPS N.V., Eindhoven (NL)

(72) Inventors: Olga STAROBINETS, NEWTON, MA (US); Sandeep Madhukar DALAL, WINCHESTER, MA (US); Ranjith Naveen TELLIS, TEWKSBURY, MA (US); Yuechen QIAN, LEXINGTON, MA (US)

(21) Appl. No.: 18/283,836

(22) PCT Filed: Mar. 23, 2022
(86) PCT No.: PCT/EP2022/057561

§ 371 Date: Sep. 25, 2023

Related U.S. Application Data
(60) Provisional application No. 63/168,327, filed on Mar. 31, 2021.

(51) Int. Cl. G16H 40/20; G16H 80/00

(52) U.S. Cl. CPC G16H 40/20; G16H 80/00

(57) ABSTRACT

A remote assistance method (100) includes: applying a likelihood estimation model (42) to determine likelihoods of needing remote expert assistance for scheduled medical imaging examinations based on information on the scheduled medical imaging examinations; applying a load-balancing optimization model (44) to assign remote experts to the scheduled medical imaging examinations of the examination schedule based on the determined likelihoods of needing remote expert assistance and information on the remote experts; providing a remote assistance interface (28, 28′) via which a local operator (LO) performing a scheduled medical imaging examination can receive remote assistance from a remote expert (RE); and initiating a remote assistance session via the remote assistance interface for the scheduled medical imaging examination being performed, wherein the initiating includes automatically connecting the local operator with the remote expert assigned to the scheduled medical imaging examination being performed.

The following relates generally to the imaging arts, remote imaging assistance arts, remote imaging examination monitoring arts, imaging examination scheduling management arts, and related arts.

### BACKGROUND

The demand for high quality medical imaging by techniques such as magnetic resonance imaging (MRI), transmission computed tomography (CT), positron emission tomography (PET), and other medical imaging modalities is high, and is expected to increase with an aging population in many countries and other factors such as improved imaging system capabilities and improved techniques for generating actionable clinical findings from medical images. The increasing problem of getting highly qualified staff (sometimes referred to as imaging technicians or technologists) for performing complex medical imaging examinations has driven the concept of bundling medical expertise in remote service centers. The basic idea is to provide virtual availability of Senior Technologists as on-call remote experts in case a (local, on-site) technologist or operator performing a medical imaging examination needs assistance with a scheduled examination or runs into unexpected difficulties. In either case, the remote expert would remotely assist the on-site operator by receiving real-time views of the situation by way of screen mirroring of the display of the medical imaging device controller and optionally other information feeds such as one or more video feeds of the imaging bay. The remote expert typically would not directly operate the medical imaging device, but would provide advice or other input for assisting the local technologist by way of telephonic or videoconferencing communication.

The following discloses certain improvements.

### SUMMARY

In one aspect, a non-transitory computer readable medium stores instructions executable by at least one electronic processor to perform a remote assistance method. The method includes: receiving an examination schedule comprising scheduled medical imaging examinations and including information on the scheduled medical imaging examinations; receiving information on remote experts; applying a likelihood estimation model to determine likelihoods of needing remote expert assistance for the scheduled medical imaging examinations based on the information on the scheduled medical imaging examinations; applying a load-balancing optimization model to assign remote experts to the scheduled medical imaging examinations of the examination schedule based on the determined likelihoods of needing remote expert assistance and the information on the remote experts; providing a remote assistance interface via which a local operator performing a scheduled medical imaging examination can receive remote assistance from a remote expert; and initiating a remote assistance session via the remote assistance interface for the scheduled medical imaging examination being performed, wherein the initiating includes automatically connecting the local operator with the remote expert assigned to the scheduled medical imaging examination being performed.

In another aspect, a non-transitory computer readable medium stores instructions executable by at least one electronic processor to perform a remote assistance method. The method includes: receiving an examination schedule comprising scheduled medical imaging examinations including information on the scheduled medical imaging examinations; receiving information on remote experts; applying a likelihood estimation model to determine likelihoods of needing remote expert assistance for the scheduled medical imaging examinations based on the information on the scheduled medical imaging examinations; applying a load-balancing optimization model to assign remote experts to the scheduled medical imaging examinations of the examination schedule based on the determined likelihoods of needing remote expert assistance and the information on the remote experts by: initially assigning the remote experts to the scheduled medical imaging examinations of the examination schedule; simulating a work shift schedule of the initially assigned remote experts handling the examination schedule; calculating one or more key performance indicators (KPIs) from results of the simulating; and optimizing the assignments of the remote experts to the scheduled medical imaging examinations based on the one or more KPIs; providing a remote assistance interface via which a local operator performing a scheduled medical imaging examination can receive remote assistance from a remote expert; and initiating a remote assistance session via the remote assistance interface for the scheduled medical imaging examination being performed by automatically connecting the local operator with the remote expert assigned to the scheduled medical imaging examination being performed.

In another aspect, a remote assistance method includes: receiving an examination schedule comprising scheduled medical imaging examinations including information on the scheduled medical imaging examinations; receiving information on remote experts; applying a reinforcement learning (RL) model to determine likelihoods of needing remote expert assistance for the scheduled medical imaging examinations based on the information on the scheduled medical imaging examinations; applying a load-balancing optimization model to assign remote experts to the scheduled medical imaging examinations of the examination schedule based on the determined likelihoods of needing remote expert assistance and the information on the remote experts; providing a remote assistance interface via which a local operator performing a scheduled medical imaging examination can receive remote assistance from a remote expert; and initiating a remote assistance session via the remote assistance interface for the scheduled medical imaging examination being performed by automatically connecting the local operator with the remote expert assigned to the scheduled medical imaging examination being performed.

One advantage resides in providing a remote expert or radiologist assisting a technician with a manageable examination assistance schedule.

Another advantage resides in using models to assign a remote expert to assist a local operator with an imaging examination.

Another advantage resides in using simulations to assign a remote expert to assist a local operator with an imaging examination.

Another advantage resides in limiting examination handoffs between remote experts to assist a local operator with an imaging examination.

Another advantage resides in providing effective handoffs of medical imaging examinations from one remote expert to another to provide continuous for a local operator performing the medical imaging examination.

Another advantage resides in improving speed and quality of imaging examinations.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

FIGS. 3-5 shows example models used by the apparatus of FIG. 1.

### DETAILED DESCRIPTION

As previously noted, the concept of bundling medical expertise in remote service centers has numerous advantages. To make such a remote service center commercially viable, however, it would be advantageous to enable the remote expert to concurrently assist (or be on call to assist) a number of different local technologists performing possibly concurrent medical imaging examinations. The local technologists may be located in a single hospital, or may be distributed among several hospitals in the same geographic area (e.g., a single city) or across a larger geographical area (e.g., spread across several states or even different countries). Preferably, the remote service center would be able to connect the expert to imaging systems of different models and/or manufactured by different vendors, since many hospitals maintain a heterogeneous fleet of imaging systems. This can be achieved by screen sharing or screen mirroring technologies that provide the remote expert a real-time copy of the imaging device controller display, optionally along with video cameras to provide views of the imaging bay and, optionally, the interior of the bore or other examination region of the imaging device. Such scalability can enable many local operators to benefit from the assistance of a single highly qualified remote expert (or small group of highly qualified remote experts) in a cost-effective manner.

In order for such remote services to be successful in supporting high quality imaging operations, the load placed on the shoulders of the remote experts has to be manageable and thus well balanced. Remote experts may be expected to monitor multiple exams taking place at the same time, including examinations performed using imaging devices from different vendors, different expertise levels of the local technicians, different imaging protocols, and, depending on the scope of service provided by the remote service center, potentially even different imaging modalities (e.g., CT, MRI, PET, etc.). Such division of attention is extremely challenging as is; however, if on top of overseeing multiple scans, several of these exams are challenging and require close oversight by the remote expert—the task of meaningful support and guidance becomes nearly impossible.

In view of the foregoing, the following relates to a remote assistance system for assisting local imaging technicians in performing medical imaging examinations. Such a remote assistance system is sometimes referred to as a Radiology Operations Command Center (ROCC), and provides remote “supertech” assistance to a local technician performing an imaging examination. The ROCC may for example provide vendor- and model-agnostic screen sharing of the imaging device console with the remote expert (i.e., the “supertech”), along with telephonic and/or videoconferencing capability. Other information feeds to the remote expert may also be provided, such as a bay camera for providing the remote expert with a view of the imaging bay preferably capturing the patient loading/unloading area and/or other critical area(s), patient vital sign readings (if monitored during the imaging examination), and/or so forth.

The ROCC provides an infrastructure via which remote experts can be on-call to assist local imaging technicians during difficult portions of imaging examinations. For maximum efficiency, each remote expert on staff at any given time may be assigned to be on call for several imaging examinations in progress at the same time since any given imaging examination is unlikely to need remote expert assistance.

The workload should be balanced amongst the on-call remote experts. It is recognized herein that the likelihood that any particular imaging examination will need the assistance of a remote expert depends on a number of factors. In view of this, the following discloses various models for assigning a likelihood that an imaging examination will require remote expert assistance. These likelihoods are then used in the load balancing.

A first illustrative remote expert assistance likelihood model disclosed herein is rules-based, and makes various assumptions. The rules employed in the first model may optionally operate on information retrieved from the Hospital Information System or other database. Since this first model operates on available data, it is usable when the ROCC is first installed.

A second illustrative remote expert assistance likelihood model disclosed herein is a machine learning (ML) model that is trained on historical ROCC data to predict the likelihood of needing assistance. Since this model relies upon historical ROCC data, it may be difficult to be deployed immediately—however, as it will be trained on historical data for the specific ROCC installation, it is likely to be more accurate than the first model. A reinforcement learning (RL) model is a suitable framework for this ML model. RL can advantageously dynamically adapt as new information is received, as will be the case during ongoing ROCC operations in which the accuracy of the predicted likelihood of needing assistance is shortly confirmed or corrected when the examination is performed. Additionally, as some local technologists or operators gain experience and require less support, or as more complex protocols are introduced creating higher demand for oversight, the model will dynamically adjust to such conditions. In one variant embodiment, the first (rules based) model is initially used while ROCC data are collected for training the second (ML) model. In addition, other prediction model(s) can be used.

The following discloses two illustrative use cases for the load balancing. In a first illustrative use case, a simulator is applied to simulate workflow for a (real or synthetic) work shift schedule, and various key performance indicators (KPIs) such as scanner utilization, patient wait time, etc., are calculated. A Discrete Event Simulation (DES) is one suitable type of simulator for simulating the work shift schedule workflow. This use case, for example, enables hospital administrators to run “what if?” scenarios to optimize the design of the ROCC, such as to estimate how many remote experts should be on duty for each work shift to provide effective and efficient assistance to local imaging technicians.

A second illustrative use case is load balancing, for assigning scheduled imaging examinations to specific remote experts. In this use case, an optimization model receives the imaging examination schedules for the supported radiology laboratories, along with the likelihood that each examination will require remote expert assistance as predicted by the remote expert assistance likelihood model(s). The optimization model further receives information about the available remote experts, such as the imaging modalities/imaging system vendors on which each remote expert is qualified. The remote expert information may optionally also receive historical performance data for each remote expert, indicating how many concurrent imaging examinations each expert can handle. The load balancing optimization can be performed before each work shift, and optionally can also be run dynamically during the work shift to account for changes over time such as imaging examinations that run long.

The load balancing optimization model may employ various constraints (e.g., each expert is assigned no more than 3 concurrent imaging examinations) and may employ weighted averaging, such as, for example, weighting each assigned examination by its likelihood of needing assistance.

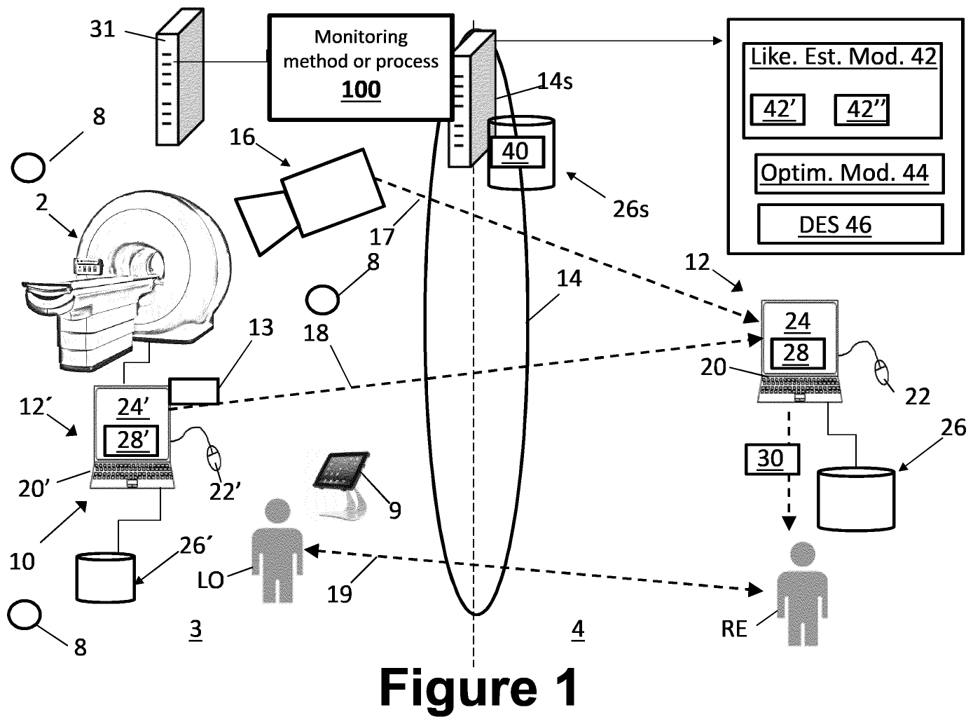

With reference to FIG. 1, an apparatus for providing assistance from a remote medical imaging expert RE (or supertech) to a local technician operator LO is shown. While a single remote expert RE and single local operator LO are shown for illustrative purposes, it will be appreciated that the remote expert RE is typically assigned (as disclosed herein) to a number of different imaging examinations, some of which may be scheduled to be performed concurrently, and which are typically scheduled to be performed by different local operators. Moreover, there may be a number of remote experts that are assigned to various scheduled imaging examinations in a load-balancing fashion as disclosed herein. As shown in FIG. 1, the illustrative local operator LO, who operates one or more medical imaging devices (also referred to as an image acquisition device, imaging device, and so forth) 2, is located in a medical imaging device bay 3, and the illustrative remote operator RE is disposed in a remote service location or center 4. It should be noted that the “remote operator” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote operator RE cannot directly visually observe the imaging device 2 in the imaging device bay 3 (hence optionally providing a video feed or screen-sharing process as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid medical imaging device such as a PET/CT or SPECT/CT imaging system. Again, while a single image acquisition device 2 is shown by way of illustration in FIG. 1, more typically a medical imaging laboratory will have multiple image acquisition devices, which may be of the same and/or different imaging modalities. Moreover, the remote service center 4 may provide service to multiple hospitals, and a single remote expert RE may concurrently monitor and provide assistance (when required) for multiple imaging bays being operated by multiple local operators, only one of which local operator is shown by way of representative illustration in FIG. 1. The local operator controls the medical imaging device 2 via an imaging device controller 10. The remote operator is stationed at a remote workstation 12 (or, more generally, an electronic controller 12). And again, the service center 4 typically has multiple remote experts on staff at a given time, who are assigned to handle imaging examinations in a load-balanced fashion as disclosed herein.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote operator RE and local operator LO ends for electronic data communications.

A screen mirroring data stream 18 is generated by a screen sharing or capture device 13, and is sent from the imaging device controller 10 to the remote workstation 12. The screen mirroring data stream 18 is provided by a screen sharing or capture device 13, which in some embodiments is a DVI splitter, a HDMI splitter, and so forth that provides a split of the DVI feed from the medical imaging device controller 10 to an external display monitor of the medical imaging device controller 10. In other embodiments, the live video feed 17 may be provided by a video cable connecting an auxiliary video output (e.g., aux vid out) port of the imaging device controller 10 to the remote workstation 12 of the operated by the remote expert RE. In yet other embodiments, the screen sharing or capture device 13 is embodied by the medical imaging device controller 10 itself running screen-sharing software. The screen mirroring data stream 18 is sent to the remote workstation 12 via the communication link 14 (e.g., as a streaming video feed received via a secure Internet link).

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is also sent to the remote workstation 12 via the communication link 14 (e.g., as a streaming video feed received via a secure Internet link).

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18 (e.g., the natural language communication pathway 19 may be provided via a landline telephone). In another example, the natural language communication pathway 19 may be provided via an ROCC device 9, such as a mobile device (e.g., a tablet computer or a smartphone). For example, an “app” can run on the ROCC device 9 (operable by the local operator LO) and the remote workstation 12 (operable by the remote expert RE) to allow communication (e.g., audio chats, video chats, and so forth) between the local operator and the remote expert.

In some embodiments, one or more sensors 8 can additionally or alternatively be disposed in the medical imaging bay 3. The sensor(s) 8 are configured to collect data related to the events corresponding to the movement of the patient or medical personnel, in addition to the number of people, in the medical imaging bay 3. In one particular example, the sensor(s) 8 can include a radar sensor configured to detect persons in the medical imaging bay 3 containing the medical imaging device 2. The radar sensor could be in addition to, or in place of, the video camera 16.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen from the screen capture device 13. Additionally or alternatively, the remote workstation 12 can be embodied as a server computer or a plurality of server computers (e.g., interconnected to form a server cluster, cloud computing resource, or so forth). The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the workstation 12. The display device 24 may also comprise two or more display devices, e.g., one display presenting the video 17 and the other display presenting the shared screen of the imaging device controller 10 generated from the screen mirroring data stream 18. Alternatively, the video and the shared screen may be presented on a single display in respective windows. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the medical imaging device bay 3 also includes similar components as the remote workstation 12 disposed in the remote service center 4. Except as otherwise indicated herein, features of the medical imaging device controller 10, which includes a local workstation 12′, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 or the mirroring data stream 18 and displayed on the remote workstation display 24 at the remote location 4.

FIG. 1 shows an illustrative local operator LO, and an illustrative remote expert RE (i.e., expert, e.g., supertech). However, in a Radiology Operations Command Center (ROCC) as contemplated herein, the ROCC provides a staff of supertechs who are available to assist local operators LO at different hospitals, radiology labs, or the like. The ROCC may be housed in a single physical location, or may be geographically distributed. For example, in one contemplated implementation, the remote operators RO are recruited from across the United States and/or internationally in order to provide a staff of supertechs with a wide range of expertise in various imaging modalities and in various imaging procedures targeting various imaged anatomies. In view of this multiplicity of local operators LO and multiplicity of remote operators RO, the disclosed communication link 14 includes a server computer 14s (or a cluster of servers, cloud computing resource comprising servers, or so forth) which is programmed to establish connections between selected local operator LO/remote expert RE. For example, if the server computer 14s is Internet-based, then connecting a specific selected local operator LO/remote expert RE can be done using Internet Protocol (IP) addresses of the various components 16, 10, 12, 8, 9, the telephonic or video terminals of the natural language communication pathway 19, etc. The server computer 14s is operatively connected with a one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s. In addition, the non-transitory computer readable medium 26s (or another database) stores data related to a set of remote experts RE and/or a set of local operators LO. The remote expert data can include, for example, skill set data, work experience data, data related to ability to work on multi-vendor modalities, data related to experience with the local operator LO and so forth. Moreover, the server computer 14s can in communication with one or more patient databases 31, including for example, a radiology information system (RIS) database, a Picture Archiving and Communication System (PACS) database, an electronic health record (EHR) database, an electronic medical record (EMR) database, and so forth.

Furthermore, as disclosed herein the server 14s performs a method or process 100 of providing remote monitoring of a local operator LO of the medical imaging device 2 during a medical imaging examination. The non-transitory computer readable medium 26s of the server computer 14s can store instructions executable by the server computer to perform the method 100 of providing remote monitoring of the local operator LO of the medical imaging device 2 during a medical imaging examination.

In addition, the non-transitory computer readable medium 26s of the server computer 14s can store an examination schedule 40 of examinations to be performed by the local operator LO, with possible assistance from the remote expert. The scheduled medical imaging examinations include information on the scheduled medical imaging examinations. The examination schedule 40 can be transferred to the server computer 14s from a central processing center (not shown) or from one of the remote experts (i.e., via the remote workstation 12). In addition, the server computer 14s can store information on the remote experts, such as historical remote assistance performance data related to the remote expert RE, along with other remote experts who are to assist the local operator LO with examinations on the examination schedule 40.

Moreover, the server computer 14s is programmed to implement one or more models. In some embodiments, a likelihood estimation model 42 is implemented by the server computer 14s to determine likelihoods of needing remote expert assistance for the scheduled medical imaging examinations based on the information on the scheduled medical imaging examinations. In one example, the likelihood estimation model 42 comprises a rules-based model 42′. In another example, the likelihood estimation model 42 comprises a machine-learning (ML) model 42″. The ML model 42″, for example, can be trained on historical data related to the remote expert RE, and information from a database (e.g., the patient database 31). In some embodiments, the ML model 42″ is a reinforcement learning (RL) model.

Furthermore, a load-balancing optimization model 44 is provided to assign remote experts to the scheduled medical imaging examinations of the examination schedule. In some examples, the load-balancing optimization model 44 may employ weighted averaging, such as weighting each assigned examination by its likelihood of needing assistance and adjusting the examination assignments amongst the on-duty remote experts using a load metric L_{RE} for each remote expert such as L_{RE}=Σ_{e∈{E}}(Pₗ)ₑ where {E} is the set of imaging exams assigned to a particular remote expert and (Pₗ), is the likelihood of needing assistance (from the likelihood estimation model 42) for the examination indexed by e. The load-balancing optimization model 44 then adjusts the distribution of the examinations amongst the on-duty remote experts to minimize L_{RE} for the various remote experts. The load-balancing optimization model 44 may additionally or alternatively employ various constraints. For example, each remote expert may be constrained to be assigned no more than 3 concurrent imaging examinations, and/or no remote expert has L_{RE} greater than some constraint limit. The constraints may also be tailored for specific remote experts, such as the constraint on the maximum number of concurrent imaging examinations a given remote expert may be assigned may be set differently for different remote experts based on information on the remote experts such as their expertise, seniority level, or so forth). Alternatively or additionally, a simulation-based optimization approach could be introduced: maximizing staff utilization, minimizing likelihood of ad-hoc assistance requests, minimizing patient wait time and etc.

In further embodiments, a Discrete Event Simulation (DES) simulator 46 can be implemented on the server computer 14s to simulate a work schedule of the remote experts handling the examination schedule the examinations on the examination schedule 40.

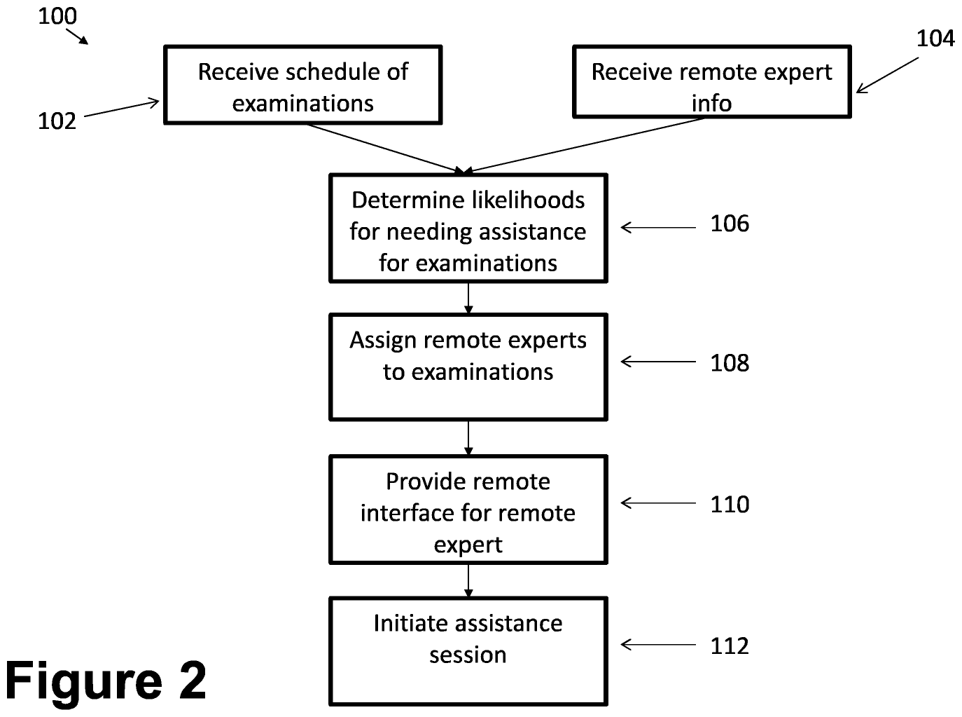

With reference to FIG. 2, and with continuing reference to FIG. 1, an illustrative embodiment of the monitoring method 100 is diagrammatically shown as a flowchart. To begin the monitoring method 100, at an operation 102, the examination schedule 40 is received at the server computer 14s. At an operation 104, the information about the remote experts, such as historical remote assistance performance data related to the remote expert RE, along with other remote experts who to assist the local operator LO with examinations on the examination schedule 40, is received at the server computer 14s.

At an operation 106, the likelihood estimation model 42 is applied to determine likelihoods of needing remote expert assistance for the scheduled medical imaging examinations based on the information on the scheduled medical imaging examinations. In some embodiments, for each scheduled medical imaging examination, the likelihood estimation model 42 comprises the rules-based model 42′ and is applied to the information on the scheduled medical imaging examination to determine the likelihood of needing remote expert assistance for the scheduled medical imaging examinations.

In another embodiment, for each scheduled medical imaging examination, the likelihood estimation model 42 comprises the ML, model 42″ and is applied to the information on the scheduled medical imaging examination to determine a likelihood of needing remote expert assistance for the scheduled medical imaging examination. In this embodiment, prior to the applying, the ML model 42″ can be trained.

At an operation 108, the load-balancing optimization model 44 is applied to assign remote experts to the scheduled medical imaging examinations of the examination schedule based on the determined likelihoods of needing remote expert assistance and the information on the remote experts. In some examples, the applying of the load-balancing optimization model 44 occurs before a workshift in which the scheduled medical imaging examinations of the examination schedule 40 are performed. In some examples, the applying of the load-balancing optimization model 44 occurs during a workshift (i.e., in real time) in which the scheduled medical imaging examinations of the examination schedule 40 are performed.

In some embodiments, the applying of the load-balancing optimization model 44 can include various processes. First, the remote experts RE are initially assigned to the scheduled medical imaging examinations of the examination schedule 40. A work shift schedule of the initially assigned remote experts handling the examination schedule 40 is simulated using the DES simulator 46. One or more key performance indicators (KPIs) are calculated from results of the simulating. The assignments of the remote experts RE to the scheduled medical imaging examinations on the examination schedule 40 based on the one or more KPIs.

At an operation 110, a remote assistance interface is provided via the GUI 28 of the remote workstation 12, and the GUI 28′ of the local workstation 12′. Via the remote assistance interface, the local operator LO can receive remote assistance from the remote expert RE during a medical imaging examination performed by the local operator using a medical imaging device 2.

At an operation 112, a remote assistance session is initiated via the remote assistance interface 28, 28′ for the scheduled medical imaging examination being performed by automatically connecting the local operator LO with the remote expert RE assigned to the scheduled medical imaging examination being performed. This can be performed by stablishing the natural communication pathway 19 between the local operator LO and the remote expert RE.

In one contemplated variant, the information on the remote experts may include information provided by the remote experts, such as a desired workload. For example, a remote expert may self-specify the maximum number of concurrent examinations he or she wishes to be assigned, and this may then be a constraint of the load-balancing optimization model 44. As another example, if the service center provides remote assistance for multiple imaging modalities, different types of imaging examinations, or so forth, then each remote expert may have the option of choosing which imaging modalities, types of imaging examinations, or so forth, they are willing to handle, and this information can again be used in the load-balancing optimization model 44. To this end, in such variant embodiments the GUI 28 of the remote workstation 12 suitably provides a configuration user dialog via which the remote expert enters the self-specified information.

### Examples

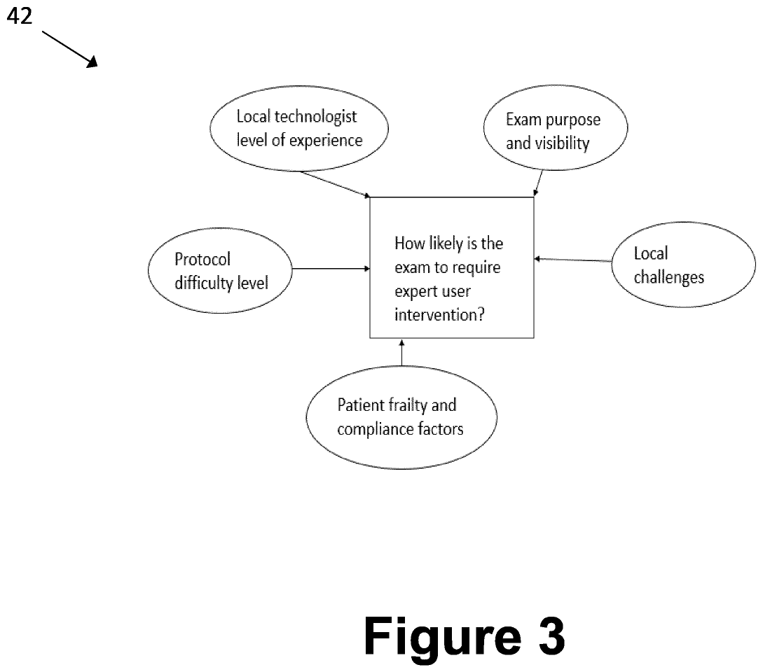

FIG. 3 shows an example of the likelihood estimation model 42. The likelihood estimation model 42 uses a set of features to predict whether an imaging examination is likely to require expert user intervention. When the ROCC system is first introduced into the imaging workflow of an organization, there is no data on how the users interact with the system, which scans require attention, which users require guidance, etc. However, by making reasonable assumptions, using retrospective operational data, and understanding skills and limitations of the local operators LO, it is possible to predict the likelihood that expert user involvement would be warranted. The set of features can include, for example, a level of protocol difficulty (while certain types of exams are commonplace, others are less common, more involved and often more difficult for inexperienced technologists to carry out well); a level the remote expert's RE experience (more experienced remote experts RE are less likely to require remote tech oversight and guidance); patient characteristics (some exam challenges may stem from the type of patient being scanned; inexperienced local operators LO may require help when scanning frail, claustrophobic, pediatric, etc. patients); examination purpose (the history or purpose of the exam may also play a role in whether expert user is likely to get involved. An exam done for a recall patient is more likely to involve expert user assistance than a first visit exam. A scan done for planning of interventions or evaluations of administered therapy typically face more scrutiny and are more likely to require additional oversight); updated software, new scanner installations, new coils or protocol changes could also contribute to remote technologist intervention, and so forth. The likelihood estimation model 42, for example, can determine a nature of the exam (i.e., challenging cardiac scan), experience level of the local operator LO (i.e., 1-2 years' experience), patient characteristics (claustrophobic patient) there is an 80% chance that the local operator LO will require assistance, it would not be appropriate for the system to assign multiple such exams taking place at the same time to the same remote expert RE.

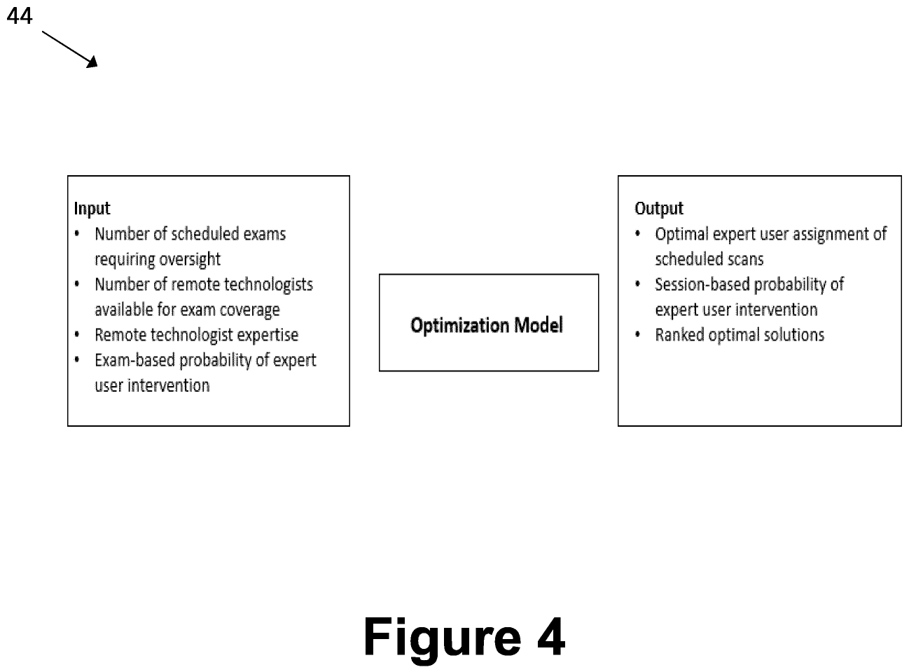

FIG. 4 shows an example of the load-balancing optimization model 44. The load-balancing optimization model 44 is implemented to ensure that a remote expert RE will not be scheduled with two or three challenging exams taking place simultaneously and requiring attention. The load-balancing optimization model 44 considers probabilities for remote expert RE involvement and identify optimal combinations for assignment of scheduled exams, ensuring a mix of cases likely requiring active involvement and those needing passive oversight.

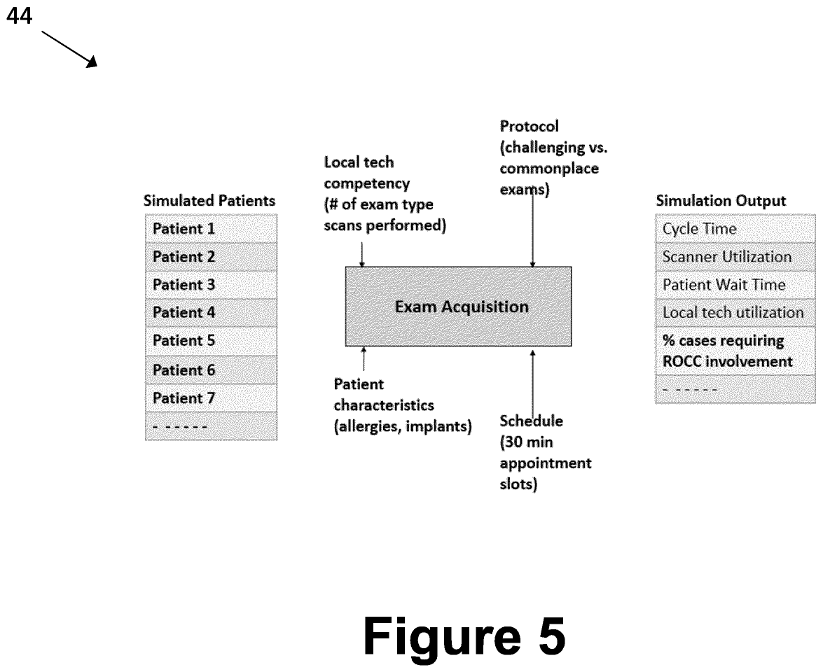

FIG. 5 shows an example of the DES simulator 46. The DES simulator 46 uses institutional data (e.g., distribution of scans, numbers, and experience levels of local operators LO, scheduling approaches, etc.) to be used to simulate demands on remote expert RE time (e.g., determine the number of scans each expert user can be expected to oversee at a given time).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a remote assistance method comprising:
receiving an examination schedule comprising scheduled medical imaging examinations and including information on the scheduled medical imaging examinations;
receiving information on remote experts;
applying a likelihood estimation model to determine likelihoods of needing remote expert assistance for the scheduled medical imaging examinations based on the information on the scheduled medical imaging examinations;
applying a load-balancing optimization model to assign remote experts to the scheduled medical imaging examinations of the examination schedule based on the determined likelihoods of needing remote expert assistance and the information on the remote experts;
providing a remote assistance interface via which a local operator performing a scheduled medical imaging examination can receive remote assistance from a remote expert; and
initiating a remote assistance session via the remote assistance interface for the scheduled medical imaging examination being performed, wherein the initiating includes automatically connecting the local operator with the remote expert assigned to the scheduled medical imaging examination being performed.

2. The non-transitory computer readable medium of claim 1, wherein the applying of the likelihood estimation model to determine the likelihoods of needing remote expert assistance for the scheduled medical imaging examinations includes:
for each scheduled medical imaging examination, applying a rules-based model to the information on the scheduled medical imaging examination to determine the likelihood of needing remote expert assistance for the scheduled medical imaging examinations.

3. The non-transitory computer readable medium claim 1, wherein the applying of the likelihood estimation model to determine the likelihoods of needing remote expert assistance for the scheduled medical imaging examinations includes:
for each scheduled medical imaging examination, applying a machine-learning model to the information on the scheduled medical imaging examination to determine a likelihood of needing remote expert assistance for the scheduled medical imaging examination.

4. The non-transitory computer readable medium of claim 3, wherein the method further comprises:
training the ML model on historical data related to the remote expert and retrieved from a database.

5. The non-transitory computer readable medium claim 3, wherein the ML model is a reinforcement learning model.

6. The non-transitory computer readable medium claim 1, wherein the applying of the load-balancing optimization model to assign remote experts to the scheduled medical imaging examinations of the examination schedule comprises:
initially assigning the remote experts to the scheduled medical imaging examinations of the examination schedule;
simulating a work shift schedule of the initially assigned remote experts handling the examination schedule;
calculating one or more key performance indicators from results of the simulating; and
optimizing the assignments of the remote experts to the scheduled medical imaging examinations based on the one or more KPIs.

7. The non-transitory computer readable medium of claim 6, wherein the simulating is performed with a Discrete Event Simulation (DES) simulator.

8. The non-transitory computer readable medium of claim 1, wherein:
the information on the remote experts includes historical remote assistance performance data related to the remote expert.

9. The non-transitory computer readable medium of claim 1, wherein the applying of the load-balancing optimization model occurs before a workshift in which the scheduled medical imaging examinations of the examination schedule are performed.

10. The non-transitory computer readable medium of claim 1, wherein the applying of the load-balancing optimization model occurs during a workshift in which the scheduled medical imaging examinations of the examination schedule are performed.

11. A non-transitory computer readable medium storing instructions executable by at least one electronic processor to perform a remote assistance method comprising:
receiving an examination schedule comprising scheduled medical imaging examinations including information on the scheduled medical imaging examinations;
receiving information on remote experts;
applying a likelihood estimation model to determine likelihoods of needing remote expert assistance for the scheduled medical imaging examinations based on the information on the scheduled medical imaging examinations;
applying a load-balancing optimization model to assign remote experts to the scheduled medical imaging examinations of the examination schedule based on the determined likelihoods of needing remote expert assistance and the information on the remote experts by:
initially assigning the remote experts to the scheduled medical imaging examinations of the examination schedule;
simulating a work shift schedule of the initially assigned remote experts handling the examination schedule;
calculating one or more key performance indicators from results of the simulating; and
optimizing the assignments of the remote experts to the scheduled medical imaging examinations based on the one or more KPIs;
providing a remote assistance interface via which a local operator performing a scheduled medical imaging examination can receive remote assistance from a remote expert; and
initiating a remote assistance session via the remote assistance interface for the scheduled medical imaging examination being performed by automatically connecting the local operator with the remote expert assigned to the scheduled medical imaging examination being performed.

12. The non-transitory computer readable medium of claim 11, wherein the applying of the likelihood estimation model to determine the likelihoods of needing remote expert assistance for the scheduled medical imaging examinations includes:
for each scheduled medical imaging examination, applying a rules-based model to the information on the scheduled medical imaging examination to determine the likelihood of needing remote expert assistance for the scheduled medical imaging examinations.

13. The non-transitory computer readable medium of claim 11, wherein the applying of the likelihood estimation model to determine the likelihoods of needing remote expert assistance for the scheduled medical imaging examinations includes:
for each scheduled medical imaging examination, applying a machine-learning model to the information on the scheduled medical imaging examination to determine a likelihood of needing remote expert assistance for the scheduled medical imaging examination.

14. The non-transitory computer readable medium of claim 13, wherein the method further comprises:
training the ML model on historical data related to the remote expert and retrieved from a database.

15. The non-transitory computer readable medium of claim 13, wherein the ML model is a reinforcement learning model.

16. The non-transitory computer readable medium of claim 11, wherein the simulating is performed with a Discrete Event Simulation simulator.

17. The non-transitory computer readable medium of claim 11, wherein:
the information on the remote experts includes historical remote assistance performance data related to the remote expert.

18. A remote assistance method comprising:
receiving an examination schedule comprising scheduled medical imaging examinations including information on the scheduled medical imaging examinations;
receiving information on remote experts;
applying a reinforcement learning model to determine likelihoods of needing remote expert assistance for the scheduled medical imaging examinations based on the information on the scheduled medical imaging examinations;
applying a load-balancing optimization model to assign remote experts to the scheduled medical imaging examinations of the examination schedule based on the determined likelihoods of needing remote expert assistance and the information on the remote experts;
providing a remote assistance interface via which a local operator performing a scheduled medical imaging examination can receive remote assistance from a remote expert; and
initiating a remote assistance session via the remote assistance interface for the scheduled medical imaging examination being performed by automatically connecting the local operator with the remote expert assigned to the scheduled medical imaging examination being performed.

19. The remote assistance method of claim 18, wherein the applying of the load-balancing optimization model to assign remote experts to the scheduled medical imaging examinations of the examination schedule comprises:
initially assigning the remote experts to the scheduled medical imaging examinations of the examination schedule;
simulating a work shift schedule of the initially assigned remote experts handling the examination schedule;
calculating one or more key performance indicators from results of the simulating; and
optimizing the assignments of the remote experts to the scheduled medical imaging examinations based on the one or more KPIs.

20. The remote assistance method of claim 19, wherein the simulating is performed with a Discrete Event Simulation simulator.
